(19) United States
(12) Patent Application Publication
(10) Pub. No.: US 2024/0156732 A1
(43) Pub. Date: May 16, 2024

(54) Composition for Liposomal Delivery of Supplemental Creatine

(71) Applicant: Spartacus Brands LLC, Los Angeles, CA (US)

(72) Inventors: Augustin Quancard, Los Angeles, CA (US); Gregory Papigny, Los Angeles, CA (US); Jeremy Eisenberg, Los Angeles, CA (US); Stephanie Eisenberg, Los Angeles, CA (US)

(21) Appl. No.: 17/988,570

(22) Filed: Nov. 16, 2022

(51) Int. Cl. A61K 9/127; A61K 31/205

(52) U.S. Cl. CPC A61K 9/127; A61K 31/205; A61K 9/0053

(57) ABSTRACT

A composition configured for oral consumption and formulated to improve the delivery of supplemental creatine is provided. The composition comprises supplemental creatine and a liposomal blend configured to encapsulate the supplemental creatine. Encapsulating the supplemental creatine with the liposomal blend improves the bioavailability of the supplemental creatine by protecting the supplemental creatine from degradation caused by the user's digestive system. The liposomal blend may further bind to the user's small intestine permitting the supplemental creatine to pass directly into the user's bloodstream, where it is accessible to the user's muscles.

### GOVERNMENT CONTRACT

Not applicable.

### CROSS-REFERENCE TO RELATED APPLICATIONS

Not applicable.

### STATEMENT RE. FEDERALLY SPONSORED RESEARCH/DEVELOPMENT

Not applicable.

### COPYRIGHT & TRADEMARK NOTICES

A portion of the disclosure of this patent document may contain material which is subject to copyright protection. This patent document may show and/or describe matter which is or may become trade dress of the owner. The copyright and trade dress owner has no objection to the facsimile reproduction by any one of the patent document or the patent disclosure, as it appears in the Patent and Trademark Office patent files or records, but otherwise reserves all copyrights and trade dress rights whatsoever.

### TECHNICAL FIELD

The disclosed subject matter relates generally to drug delivery and more particularly to targeted drug delivery of ingestible supplemental creatine using liposomes.

### BACKGROUND

Dietary supplements have become increasingly popular in recent years for a variety of reasons, including the support of muscle function, strength, and to supplement nutritional deficiencies caused by a person's diets. For example, creatine is an amino acid created by the body's liver, pancreas, and kidneys and received through seafood and red meat, for use with muscle contraction, protein metabolism, and even cognition. However, a typical individual, without dietary restrictions or other health conditions, only produces enough creatine to fill 60-80% of their body's creatine capacity. As a result, many weight-lifters and other athletes use supplemental creatine to provide the remaining 20-40% in order to improve their muscle function.

The general form of supplemental creatine is creatine monohydrate, such as the formulation described in U.S. Patent Pub. No. 2007/0,292,403 to Nivaggioli. While some beneficial results of supplemental creatine may be seen immediately, it can take days or even weeks, called a loading period, to achieve creatine saturation in one's muscles. Once creatine saturation has been reached, benefits include increased muscle gain, strength, athletic performance, and even injury prevention, however, prior to reaching creatine saturation, there is only a limited and temporary improvement.

Though the primary use of supplemental creatine is to increase muscle performance during exercise, supplemental creatine may be used in treatment for a wide range of conditions, such as neurodegenerative diseases, diabetes, osteoarthritis, fibromyalgia, aging, brain and heart ischemia, and adolescent depression. As is the case with supplemental creatine for exercise, supplemental creatine requires a loading period to reach sufficient creatine saturation for changes to be observed.

Currently, creatine saturation requires a five to seven-day “loading period” of high-dose supplemental creatine to saturate the muscles. However, studies have shown that during this high-dose period, only 16% of the supplemental creatine is absorbed by the body. While low-dose loading periods have shown a 53% absorption rate, reducing the amount of supplemental creatine waste, it takes approximately four weeks to reach muscle saturation. Further, supplemental creatine waste does not end once muscles have been fully saturated, instead, the supplemental creatine needed to maintain saturation remains at a 53% absorption rate. As a result, a large amount of supplemental creatine is wasted because of its poor bioavailability.

One proposal to improve bioavailability of supplemental creatine is provided in U.S. Pat. No. 10,881,630 to Miller et al. for creatine hydrochloride. However, the use of creatine hydrochloride has been minimally studied, and there is little evidence to support that creatine hydrochloride provides increased bioavailability over traditional creatine monohydrates. As a result, it is unclear if Miller's proposed creatine hydrochloride is effective. Further, because of the minimal studies, many are reluctant to transition from the extensively studied creatine monohydrate to an unsupported and often more expensive supplement.

Some, such as Farber in U.S. Patent Pub. No. 2007/0196496, have proposed the delivery of nutritional supplements, including supplemental creatine, using an ingestible matrix. For example, the matrix in Faber comprises carbohydrates, sugar, hydrocolloid, solvent, and in some embodiments, bioavailability enhancers to increase the absorption of a large number of active ingredients. While Farber teaches the use of bioavailability enhancers, Farber's disclosed enhancers are commonly known enhancers in the art for a wide variety of active ingredients, such as ibuprofen. Farber does not teach any improvement to the enhancers but instead merely provides that the enhancers may be mixed with a variety of additional ingredients, including sugars, corn syrup, and gelatin. As a result, Farber does not propose improved bioavailability for supplemental creatine but instead teaches that any of the disclosed active ingredients may be combined with known bioavailability enhancers.

Indeed, poor bioavailability is a common problem for many active ingredients, and the means to improve bioavailability is unique to each. As a result, there are many proposals to increase bioavailability, such as essential oils as proposed in U.S. Pat. No. 7,716,928 to Benet, menthol as proposed by Flashner-Barak in U.S. Patent Pub. No. 2004/0,198,646, and even salts as described in U.S. Patent Pub. No. 2019/0,083,407 to Hanna. These proposals, however, introduce additives that may introduce allergens, affect the stability, or even counteract the active ingredients in another drug. In addition, since there is no universal solution for improved bioavailability of active ingredients, any proposed solution to the problem of poor bioavailability must be viewed according to its active ingredients.

Some drugs, such as chemotherapy and vaccinations, have shown increased bioavailability through the use of liposomal matrices as described in U.S. Pat. No. 9,655,846 to Javeri. Liposomal matrices operate by encapsulating the active ingredients and binding to a cell membrane to allow the active ingredients to be absorbed directly into the bloodstream. However, studies have shown that amino acids inhibit liposomal drug matrices in some instances, such as gene delivery to the lungs. Elga Bandeira et al., Association with Amino Acids Does Not Enhance Efficacy of Polymerized Liposomes as a System for Lung Gene Delivery (2016).

Although various solutions have been proposed to address the bioavailability of active ingredients, none combines the characteristics of the present invention. Thus, there remains a need to increase the bioavailability of supplemental creatine, specifically in the common creatine monohydrate form, to the user's muscles.

### SUMMARY

The present disclosure is directed to a composition for increased absorption of ingested supplemental amino acids, using liposomal drug delivery to permit targeted delivery of a supplemental amino acid to a user. More particularly, the composition comprises the supplemental amino acid and a liposomal blend operative to improve the bioavailability of the supplemental amino acid by preventing degradation caused by the digestive system and permitting the direct release of the supplemental amino acid into a targeted region.

For purposes of summarizing, certain aspects, advantages, and novel features have been described. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested.

In one embodiment, the supplemental amino acid may be creatine. In some embodiments, the supplemental amino acid may be creatine monohydrate, however, in another embodiment, it may be any form of supplemental creatine. Of course, any supplemental amino acid may be utilized including, without limitation, glutamine, lysine, branched-chain amino, or any other supplemental amino acid as needed or desired.

The liposomal blend may comprise liposomes, including, for example, and without limitation, phospholipids, egg, or any other form of liposomes. A person of ordinary skill in the art will recognize that liposomes are artificial vesicles comprising a hydrophobic membrane of at least one lipid bilayer and an aqueous core. Phospholipids, liposomes, and lipid bilayers, and in the interest of brevity, only a brief description of such components has been provided.

The phospholipids may be any phospholipid known in the art, for example, may be any of phosphatidylcholine, phosphorylethanolamine, phospholipid, or phosphatidylserine. The phospholipids may form a lipid bilayer similar to that of a cell membrane. As such, the phospholipids in the lipid bilayer may be operative to bind with the cell membrane. The phospholipids binding to the cell membrane creates an opening in the cell membrane that corresponds with an opening in the liposome to define a passage. As discussed in more detail below, the passage between the liposome and the cell membrane may permit the supplemental amino acids to pass directly into the user's bloodstream.

In some embodiments, the phospholipids may be plant-based. For example, in an embodiment wherein the phospholipid is phosphatidylcholine, the phosphatidylcholine may be extracted from sunflowers. It is contemplated that by using plant-based phospholipids, the composition may be vegetarian, or even vegan, and thus may be available to those with specific dietary needs. Of course, other forms of phospholipids may be utilized.

The supplemental amino acid may be at least partially mixed in the aqueous core of the liposomes. Thus, the liposomes in the liposomal blend may encapsulate the supplemental amino acid, thus protecting the supplemental amino acid from the digestive system, and reducing the amount of supplemental amino acid lost through digestion. Traditionally, ingested supplements are degraded prior to absorption by gastric juices that are encountered throughout the digestive system. However, liposomes comprise phospholipids that form a barrier around the supplemental amino acids to protect against this degradation, permitting the encapsulated supplemental amino acid to reach a targeted region without the effects of degradation. A person of ordinary skill will recognize that by reducing the amount of supplemental amino acid lost through digestion, more of the ingested supplemental amino acid may be absorbed by the user.

Further, the liposomes may be operative to bind to cell membranes, permitting the supplemental amino acid to diffuse across the cell membrane. As an example, and without limitation, the phospholipids may bind to a cell membrane, permitting the encapsulated supplemental amino acid to pass directly through the cell membrane and into the cell body. As previously discussed, phospholipids binding to the cell membrane define a passage. This passage may permit the supplemental amino acid to directly enter the cell body, without a loss that occurs due to the cell membrane. For example, the phospholipids may bind with the cell membrane of the small intestine, permitting the supplemental amino acid to directly pass into the user's bloodstream. The supplemental amino acid may then be absorbed by the muscles and other areas of the body. As a result, the supplemental amino acid absorbed by the muscles may be essentially all the supplemental amino acid ingested.

Indeed, a person of ordinary skill in the art will recognize the manners in which the liposomes may bind to the cell membrane. The liposomal blend being operative to bind to the cell membrane may permit the supplemental amino acid to pass directly into the targeted region of the user's body, as such, increasing absorption of the supplemental amino acid. Thus the liposomal blend may protect the supplemental amino acid from degradation that occurs due to the digestive system.

In yet another embodiment, the liposomal blend may be operative to control the release of the supplemental amino acid. It is contemplated that controlling the release of the supplemental amino acid may alter the period of time in which the supplemental amino acid is effective, permitting a more effective loading of amino acid. Further, the controlled release may permit the supplemental amino acid to have an increased half-life, extending an effective period of the supplemental amino acid.

It is further contemplated that the liposomal blend may be further operative to stabilize the composition and thus extend the shelf-life.

It is contemplated that formulating a composition for increased absorption of supplemental amino acids according to the disclosure and claims provided herein may have the following advantages:
a.) increased absorption of supplemental amino acid;
b.) improves the bioavailability of the supplemental amino acid;
c.) reduces waste of ingested supplemental amino acid caused by the digestive system;
d.) controls the release of supplemental amino acid; and
e.) increases the stability of the supplemental amino acid.

Thus, it is an object of the invention to increase the absorption of ingested supplemental amino acids.

It is yet a further object of this invention to reduce supplemental amino acid waste.

It is yet another object of this invention to increase the shelf-life of supplemental amino acids.

It is still a further object of this invention to provide for the controlled release of supplemental amino acids.

It is yet a further object of this invention to distribute supplemental amino acids to targeted areas of the body.

It is still another object of this invention to reduce the time required to reach supplemental creatine saturation.

One or more of the above-disclosed embodiments, in addition to certain alternatives, are provided in further detail below with reference to the attached figures. The disclosed subject matter is not, however, limited to any particular embodiment disclosed.

### DETAILED DESCRIPTION

Having summarized various aspects of the present disclosure, reference will now be made in detail to that which is illustrated in the drawings. While the disclosure will be described in connection with these drawings, there is no intent to limit it to the embodiment or embodiments disclosed herein. Rather, the intent is to cover all alternatives, modifications and equivalents included within the spirit and scope of the disclosure as defined by the appended claims.

In accordance with one embodiment, the composition may comprise supplemental amino acid and a liposomal blend. The liposomal blend may comprise liposomes, which will be understood by a person of ordinary skill in the art. However, in the interest of clarity, non-limiting examples and embodiments of liposomes are discussed, though any form or embodiment of liposomes may be utilized.

In one embodiment, the composition may comprise between about 0.01 to about 10 percent by weight liposomal blend and between about 90 to about 99.99 percent by weight supplemental amino acid.

In another embodiment, the composition may comprise between about 0.05 to about 5 percent by weight liposomal blend and between about 95 to about 99.95 by weight supplemental amino acid.

In yet another embodiment, the composition may comprise about 0.99 percent by weight liposomal blend and about 99.01 percent by weight supplemental amino acid.

In one exemplary embodiment, the supplemental amino acid may comprise creatine. Any type of creatine may be utilized, including, and without limitation creatine monohydrate, creatine ethyl ester, creatine hydrochloride, buffered creatine, and creatine magnesium chelate. However, in the interest of clarity and brevity, an embodiment describing creatine monohydrate is described, however, a person of ordinary skill in the art will appreciate that any form of creatine may be utilized to carry out the disclosure.

The liposomes in the liposomal blend may be any liposomes known in the art. In general, liposomes comprise at least one lipid bilayer surrounding an aqueous core. The lipid bilayer may be formed from phospholipids, which comprise a hydrophilic head and a hydrophobic tail. Liposomes are generally spherical in shape and will be discussed as such for the sake of brevity, however, liposomes may vary in shape and form and any may be utilized. A person of ordinary skill in the art will appreciate that liposomes may contain an active ingredient, such as the supplemental amino acid, in the aqueous core or between the hydrophobic tails of the lipid bilayer. While the supplemental amino acid may be contained in either, or even both, of the aforementioned locations, in the interest of brevity, reference, is made, in particular, throughout the disclosure, to the embodiments wherein the supplemental amino acid is contained in the aqueous core.

A person of ordinary skill will appreciate that encapsulating the supplemental amino acid in the liposomes may be done in any number of manners, and the subsequent embodiments are provided as non-limiting examples only. Generally, liposome preparation will comprise drying lipids, dispersing the lipids in an aqueous media to form the liposomes, and purifying the liposome. The aqueous media may comprise the supplemental amino acid that, when encapsulated, may form the aqueous core.

In one embodiment, the supplemental amino acid may be encapsulated in the liposome through a passive loading method. For example, the passive loading method may be mechanically dispersed in the liposome through any of mechanical dispersion methods, solvent dispersion method, and detergent removal method. Of course, other forms of passive loading may be utilized and the aforementioned are provided as examples only. In the interest of clarity, one embodiment of mechanical dispersion is provided, though any form of passive encapsulation may be utilized. Mechanical dispersion may utilize a process known in the art as sonication where the lipids and aqueous media are exposed to high frequency sound waves. It is contemplated that sonication may create a more homogenous suspension and may reduce large particles.

In another embodiment, the supplemental amino acid may be encapsulated in the liposomes through active loading. Active loading may occur following the formation of liposomes and may permit the supplemental amino acid to be added at a later time. In some embodiments, active loading may be accomplished using gradient loading which involves a buffer that uses a PH gradient for loading the supplemental amino acid into the liposome. For example, the aqueous core of the liposome may comprise a low PH and may be suspended in a solution comprising a high PH and the supplemental amino acid. The solution may permeate the liposome to equalize the PH, thus delivering the supplemental amino acid into the aqueous core of the liposome.

In one embodiment, the phospholipids may be lecithin phospholipids. In a further embodiment, the lecithin phospholipids may be extracted from sunflowers. More particularly, the lecithin phospholipids may be extracted from sunflower oil. Of course, phospholipids may be extracted from a large number of plants and animals, such as from eggs, meat, seafood, and soy. As such, any form of phospholipids may be utilized, and the aforementioned are provided as non-limiting examples.

The terms “lecithin,” “phospholipids,” and “phosphatidylcholine” are used interchangeably. Indeed, a person of ordinary skill will appreciate that lecithin may comprise additional substances, such as triglycerides, fatty acids, and carbohydrates. While phospholipids and phosphatidylcholine may be generally understood to comprise a lower concentration of the additional substances, for the purposes of this disclosure, unless otherwise provided, the use of phospholipids and phosphatidylcholine is synonymous with lecithin.

In some embodiments, the composition may be ingestible. For example, the composition may be orally consumed. In one embodiment, the composition may be in a powdered form. In one such embodiment, the composition may be water soluble such that the powdered composition may be at least partially mixed in water. A person of ordinary skill will recognize that the hydrophilic heads of the liposomes in the liposomal blend may permit the liposomes to bind to the water molecules. As such, the composition may be dissolved in water to create a solution. Of course, other liquids may be utilized, such as juice, coffee, milk, or even other liquids or combinations thereof. It is contemplated that the composition may be dissolved in the same liquids as water soluble solutes, for example, electrolytes, vitamins, or flavoring.

In another embodiment, the composition may be in tablet form. In yet another embodiment, the composition may be in capsule form. Of course, the composition may be in another form, such as and without limitation to gummies, drinks, and energy bars. It is contemplated that in some embodiments, additional ingredients may be required, such as pill capsules, gelatin, or any other ingredients as needed or required.

In some embodiments, the composition may comprise flavoring. The flavoring may be present at any concentration as needed or desired. However, in other embodiments, the composition may be unflavored.

In yet another embodiment, the composition may comprise additional nutraceuticals. For example, the composition may comprise supplemental protein, vitamins, antioxidants, minerals, fibers, or any other nutraceutical that may be needed or desired. It is contemplated that the additional nutraceuticals may be present at any concentration in the composition as needed or desired.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

### EXAMPLES OF THE PREFERRED EMBODIMENT OF THE COMPOSITION

In order to more fully teach what the Applicant regards as his invention, the following example is given. It should be understood that the formulation set forth in the Example is not to be construed as limiting the scope of the invention, except so far as they yield a composition having the desired properties and characteristics. More particularly, and by way of example, the following chart illustrates a formulation of the same with the percentages given by weight of the composition.

### Example 1

| Ingredient | Percentage |
|---|---|
| Creatine Monohydrate | 99.01 |
| Liposomal Blend | 0.99 |
| TOTAL: | 100.00% |

### CONCLUSIONS, RAMIFICATIONS, AND SCOPE

While certain embodiments of the invention have been illustrated and described, various modifications are contemplated and can be made without departing from the spirit and scope of the invention. For example, the composition may comprise any active ingredient, including pharmaceuticals, vitamins, and other supplements. Accordingly, it is intended that the invention not be limited, except as by the appended claim(s).

The teachings disclosed herein may be applied to other systems, and may not necessarily be limited to any described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions and concepts of the various references described above to provide yet further embodiments of the invention.

Particular terminology used when describing certain features or aspects of the invention should not be taken to imply that the terminology is being refined herein to be restricted to any specific characteristics, features, or aspects of the composition for liposomal delivery of supplemental creatine with which that terminology is associated. In general, the terms used in the following claims should not be constructed to limit the composition for supplemental creatine to the specific embodiments disclosed in the specification unless the above description section explicitly define such terms. Accordingly, the actual scope encompasses not only the disclosed embodiments, but also all equivalent ways of practicing or implementing the disclosed system, method and apparatus. The above description of embodiments of the composition for supplemental creatine is not intended to be exhaustive or limited to the precise form disclosed above or to a particular field of usage.

While specific embodiments of, and examples for, the method, system, and apparatus are described above for illustrative purposes, various equivalent modifications are possible for which those skilled in the relevant art will recognize.

While certain aspects of the method and system disclosed are presented below in particular claim forms, various aspects of the method, system, and apparatus are contemplated in any number of claim forms. Thus, the inventor reserves the right to add additional claims after filing the application to pursue such additional claim forms for other aspects of the composition for supplemental creation.

1. A water soluble composition for oral consumption comprising: creatine monohydrate; and
a liposomal blend comprising phosphatidylcholine, wherein the liposomal blend encapsulates the creatine monohydrate to increase the absorption of the creatine monohydrate.

2. The composition of claim 1, wherein the composition comprises: about 0.99% by weight the liposomal blend; and
about 99.01% creatine monohydrate.

3. An ingestible composition for improved delivery of supplemental creatine comprising:
supplemental creatine; and
a liposomal blend.

4. The composition of claim 3, wherein the supplemental creatine comprises creatine monohydrate.

5. The composition of claim 3, wherein the liposomal blend comprises phosphatidylcholine.

6. The composition of claim 5, wherein the phosphatidylcholine is extracted from sunflowers.

7. The composition of claim 3, wherein the composition comprises from: about 0.01% to about 10% by weight liposomal blend; and
about 90% to about 99.9% by weight supplemental creatine.

8. The composition of claim 3, wherein the composition comprises from: about 0.05% to about 5% by weight liposomal blend; and
about 95% to about 99.95% by weight supplemental creatine.

9. The composition of claim 3, wherein the composition comprises from: about 0.99% by weight liposomal blend; and
about 99.01% by weight supplemental creatine.

10. The composition of claim 3, wherein the composition is for oral consumption and the liposomal blend encapsulates the supplemental creatine to increase the absorption of the supplemental creatine when ingested.

11. An ingestible composition for increased bioavailability comprising:
from about 99.99% to about 90% by weight supplemental amino acid; and
from about 1% to about 0.01% by weight a liposomal blend.

12. The composition of claim 11, wherein the supplemental amino acid comprises creatine.

13. The composition of claim 11, wherein the supplemental amino acid is creatine monohydrate.

14. The composition of claim 11, wherein the liposomal blend comprises phospholipids.

15. The composition of claim 11, wherein the liposomal blend comprises phosphatidylcholine.

16. The composition of claim 15, wherein the phosphatidylcholine is extracted from sunflowers.

17. The composition of claim 11, wherein the composition comprises from: about 0.05% to about 1% by weight the liposomal blend; and
about 95% to about 99.95% by weight supplemental amino acid.

18. The composition of claim 11, wherein the composition comprises from: about 0.99% by weight the liposomal blend; and
about 99.01% by weight supplemental amino acid.

19. The composition of claim 11, wherein the liposomal blend encapsulates the amino acid to increase absorption of the amino acid.

20. The composition of claim 11, wherein the composition is in a powdered form operative to at least partially dissolve in water.
